(19) United States
(12) Patent Application Publication
(10) Pub. No.: US 2021/0346614 A1
(43) Pub. Date: Nov. 11, 2021

(54) PORTABLE MEDICAL SPRAY

(71) Applicant: Chung-Sing LIN, Tainan (TW)

(72) Inventor: Chung-Sing LIN, Tainan (TW)

(73) Assignee: Chung-Sing LIN, Tainan (TW)

(21) Appl. No.: 15/929,533

(22) Filed: May 8, 2020

(51) Int. Cl. A61M 11/00; A61M 39/22

(52) U.S. Cl. CPC A61M 11/007; A61M 2210/0618; A61M 39/22

(57) ABSTRACT

A portable medical spray includes a tubular body having a room defined therein, and a breakable portion is formed to the top end of the body. A connection portion extends from the bottom end of the body. A nozzle is connected to the top end of the body. The nozzle has a passage which is located corresponding to the breakable portion. A medicine cell is located in the room of the body and has medicine received therein. The medicine cell is a compressible bellow cell. A needle is located in the medicine cell. An end part is mounted to the connection portion. The end part is pushed toward the nozzle to compress the medicine cell, and the needle penetrates through the top end of the medicine cell and the breakable portion to release the medicine via the nozzle.

### BACKGROUND OF THE INVENTION

### 1. Fields of the Invention

The present invention relates to a medical spray, and more particularly, to a portable medical spray.

### 2. Descriptions of Related Art

The conventional medical spray is used to soothe uncomfortable symptoms caused by rhinitis, stuffy nose, throat ache and the like, and the medical spray is inserted into the patient's nose or mouth to spray medicine onto the infected areas. When spraying the medicine, a compressor and a spray are needed to send medicine to the desired areas. These equipment may include multiple parts and occupy a lot of space. In addition, theses equipment may not be conveniently obtained in remote areas.

The present invention intends to provide a portable medicine spray to eliminate the shortcomings mentioned above.

### SUMMARY OF THE INVENTION

The present invention relates to a portable medical spray and comprises a tubular body having a room defined therein. A breakable portion is formed to the top end of the body. A connection portion extends from the bottom end of the body. A top groove and a bottom groove are respectively formed to the outside of the connection portion. A nozzle is connected to the top end of the body and has a passage defined therethrough. The passage is located corresponding to the breakable portion. A holding portion is formed to the outside of the body.

A medicine cell is located in the room and has medicine received therein. The medicine cell is a compressible bellow cell, and a needle is located in the medicine cell. An end part has a reception space defined in the top end thereof. The end part is movably mounted to the connection portion. A lip extends inward from the inner periphery of the reception space. Multiple slots are defined axially through the wall of the end part and open through the lip. The lip is removably engaged with the bottom groove when the end part is not pushed toward the nozzle. The lip is engaged with the top groove when the end part is moved toward the nozzle.

Preferably, the passage of the nozzle of the body has a valve received therein. An adjustment knob is connected to the nozzle and operates the valve.

Preferably, a cap is removably mounted to the nozzle.

The primary object of the present invention is to provide a portable medical spray which is compact for easy carry. The canister can be replaced to include different medicine therein.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is an exploded view to show the portable medical spray of the present invention;

FIG. 2 is a perspective view to show the portable medical spray of the present invention;

FIG. 3 is a cross sectional view of the portable medical spray of the present invention;

FIG. 4 is a perspective view to show the portable medical spray of the present invention, wherein the cap is removed from the nozzle;

FIG. 5 is a cross sectional view to show that the end part is pushed toward the needle and the medicine cell is compressed, and the needle penetrates through the top end of the medicine cell and the breakable portion to eject the medicine in the medicine cell from the nozzle of the portable medical spray of the present invention, and

FIG. 6 illustrates that the portable medical spray of the present invention is used as a nasal spray.

### DETAILED DESCRIPTION OF THE INVENTION

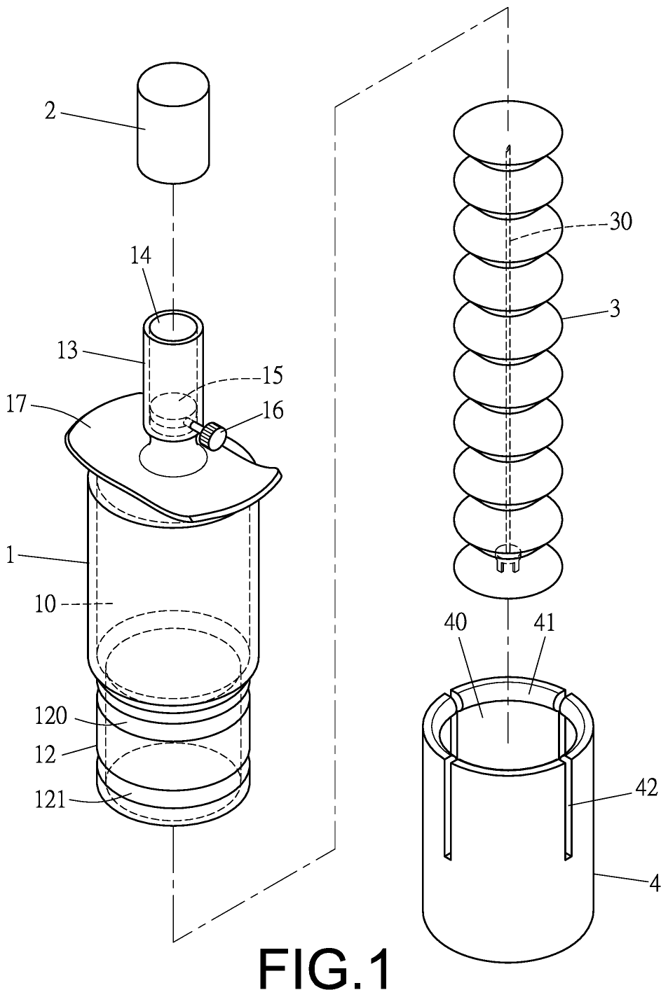

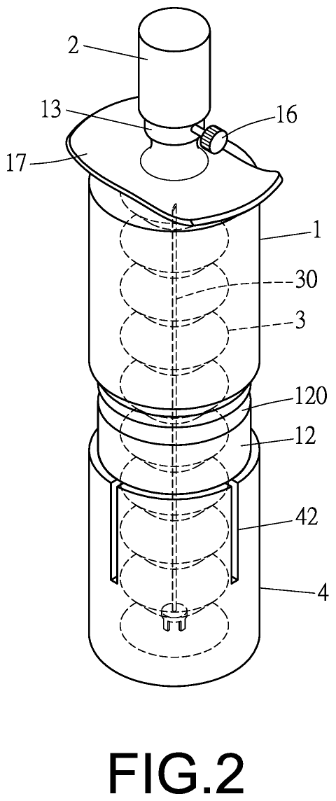

Referring to FIGS. 1 to 3, the portable medical spray of the present invention comprises a tubular body 1 having a room 10 defined therein. A breakable portion 11 is formed to the top end of the body 1. A connection portion 12 extends from the bottom end of the body 1. A top groove 120 and a bottom groove 121 are respectively formed to the outside of the connection portion 12. A nozzle 13 is connected to the top end of the body 1, and has a passage 14 defined therethrough. The passage 14 is located corresponding to the breakable portion 11. A holding portion 17 is formed to the outside of the body 1 and includes two finger flanges. The passage 14 of the nozzle 13 of the body 1 has a valve 15 received therein. An adjustment knob 16 is connected to the nozzle 13 by a rod which extends through the nozzle 12 so that the valve 15 is operated by rotating the adjustment knob 16. A cap 2 is removably mounted to the nozzle 13.

A medicine cell 3 is located in the room 10 and has medicine “A” received therein under pressure. The medicine cell 3 is a compressible bellow cell. A needle 30 is located in the medicine cell 3. An end part 4 has a reception space 40 defined in the top end thereof, and the end part 4 has a closed bottom end. The end part 4 is movably mounted to the connection portion 12 which is inserted in the reception space 40. A lip 41 extends inward from the inner periphery of the reception space 40. Multiple slots 42 are defined axially through the wall of the end part 4 and open through the lip 41. When the end part 4 is not pushed toward the nozzle 13, the lip 41 is removably engaged with the bottom groove 121 of the connection portion 12.

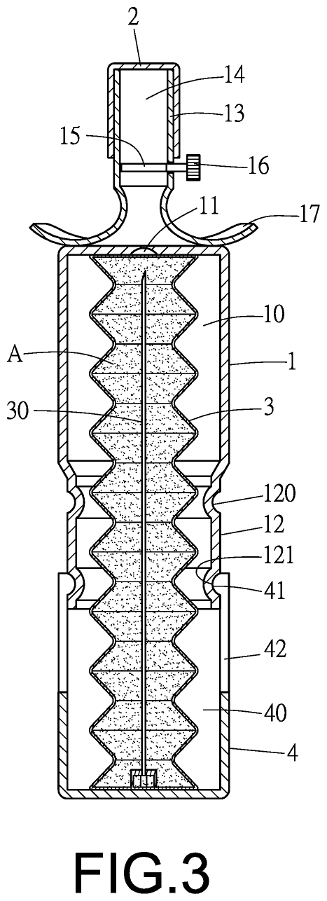

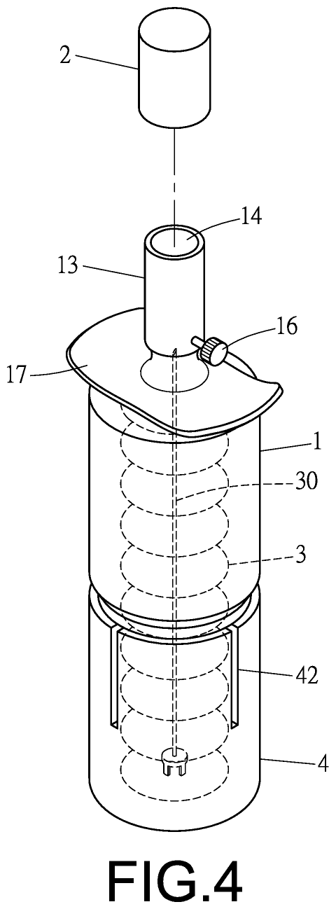

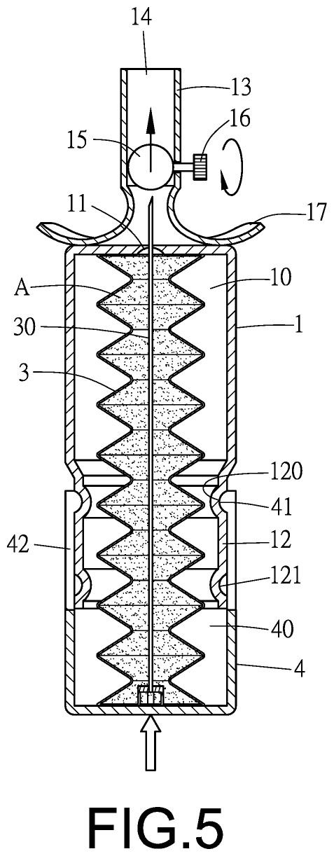

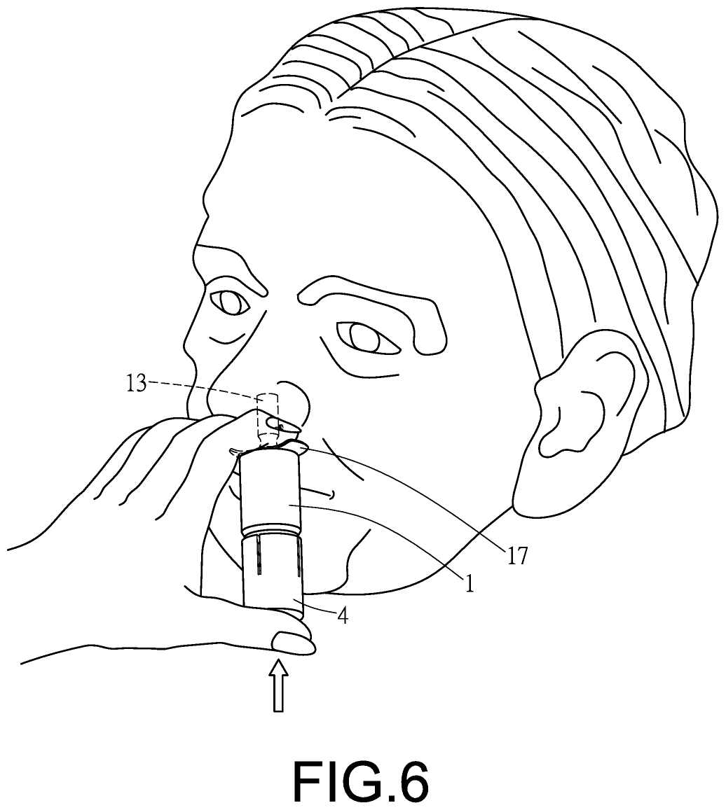

As shown in FIGS. 3 to 6, when in use, the cap 2 is removed from the nozzle 13, and the adjustment knob 16 is rotated to adjust the valve 15 to set the amount of the medicine “A” to be injected. The user uses two fingers to holds the body 1 and the two fingers put on the finger flanges of the holding portion 17. The nozzle 13 is then inserted into the patient's nose as shown in FIG. 6. The user then uses the thumb to push the end part 4 toward the needle 30 or the nozzle 13. As shown in FIG. 5, the lip 41 is removed from the bottom groove 121 and is engaged with the top groove 120 when the end part 4 is moved toward the nozzle 13. The medicine cell 3 is compressed and by the movement of the end part 4, and the needle 30 penetrates through the top end of the medicine cell 3 and the breakable portion 11 of the body 1. The medicine “A” in the medicine cell 3 then ejects into the patient's nose through the passage 14 of the nozzle 13.

The medicine cell 3 can be replaced with a new one in which different medicine “A” is stored. The portable medical spray of the present invention is compact for easy carry.

While we have shown and described the embodiment in accordance with the present invention, it should be clear to those skilled in the art that further embodiments may be made without departing from the scope of the present invention.

What is claimed is:

1. A portable medical spray comprising:
a body having a room defined therein, a breakable portion formed to a top end of the body, a connection portion extending from a bottom end of the body, a top groove and a bottom groove respectively formed to an outside of the connection portion, a nozzle connected to the top end of the body, a passage defined through the nozzle, the passage located corresponding to the breakable portion, a holding portion formed to an outside of the body;
a medicine cell located in the room and having medicine received therein, the medicine cell being a compressible bellow cell, a needle located in the medicine cell, and
an end part having a reception space defined in a top end thereof, the end part movably mounted to the connection portion, a lip extending inward from an inner periphery of the reception space, multiple slots defined axially through a wall of the end part and opening through the lip, the lip removably engaged with the bottom groove when the end part is not pushed toward the nozzle, and the lip is engaged with the top groove when the end part is moved toward the nozzle.

2. The portable medical spray as claimed in claim 1, wherein the passage of the nozzle of the body has a valve received therein, an adjustment knob is connected to the nozzle and operates the valve.

3. The portable medical spray as claimed in claim 1, wherein a cap is removably mounted to the nozzle.
